(19) United States
(12) Patent Application Publication
(10) Pub. No.: US 2006/0068396 A1
(43) Pub. Date: Mar. 30, 2006

(54) Method for biochip detecting limited cells

(75) Inventors: Shiu-Ru Lin, Kaohsiung City (TW); Tian-Lu Cheng, Kaohsiung City (TW); Yi-Fang Chen, Kaohsiung City (TW); Chan-Han Wu, Kaohsiung City (TW)

Correspondence Address: TROXELL LAW OFFICE PLLC, SUIT E1404, 5205 LEESBURG PIKE, FALLS CHURCH, VA, 22041, US

(73) Assignee: Kaohsiung Medical University

(21) Appl. No.: 10/948,224

(22) Filed: Sep. 24, 2004

(51) Int. Cl. C12Q 1/68

(52) U.S. Cl. 435/6

(57) ABSTRACT

The present invention is a method for a biochip detecting limited cells. The present invention comprises steps of: obtaining a nylon membrane chip having required nucleotide fragments arranged in a dot matrix way by using a manual spotter; naturally drying by heat and fixing the nucleotide fragments on the nylon membrane chip by a rapid nucleic acid cross-linker when preparing a chip; collecting some normal whole blood to be linearly amplified; synthesizing required amount of cDNA by a reverse transcription to obtain a marker as a probe; processing labeling, hybridization and post-hybridization to the chip and the marker; processing chemical color reaction; and automatically analyzing the result image after the chemical color reaction. Accordingly, a gene biochip operation technology platform with low cost, easy operation and high efficiency is obtained. And so the functions and the applications of the gene biochip can be effectively worked out and the practical applications of the gene biochip on related fields can be conclusively popularized.

### FIELD OF THE INVENTION

The present invention relates to a method for a biochip; more particularly, relates to a method for a gene biochip operation technology platform on which the functions and the applications of the gene biochip can be effectively worked out and the practical applications of the gene biochip on related fields can be conclusively popularized.

### DESCRIPTION OF THE RELATED ARTS

A biochip is a micro device, comprising a silicon chip, a glass or a macromolecule as a substrate to integrate organic molecules (ex. nucleic acid or protein) by minimization technology to examine or analyze bio-molecules.

Because of the small size and the rapid reaction of the biochip together with its capability in parallel analysis of a great amount of biological information, the biochip can be used in biochemical treatment, biochemical analysis, biochemical examination, new medicine investigation and environment monitoring.

One of the most complete and eye-catching research now is the biochip research, characterized in that:

A. Examine a great amount of various genes at a time: According to the different goals of the researches, the researchers can choose different nucleotide fragments to fix on chip in arrays. In general, the researchers can investigate the actions of the genes in the cell with the nucleotide fragments for rapid and mass examination. It also changes the actuality of that a researcher usually did his research of one gene only in his whole life.

B. Require less biological material: The required specimen samples, bio-probes and targets are over 20 times lesser than that of the conventional “dot blotting.”

C. Automatic analysis: Because of the support of different software for related biological information, the analysis of the results from the gene biochip can be fully automatically done. Not only two basic functions of gene expression and differential gene searching can be achieved, but also multiple applications can be derived from the mutual exercises of the two functions. These functions and applications can be applied to many fields, such as biomedical science, food examination, chemical synthesis, new medicine researches, military applications, etc.

Yet, until now, the development of the gene biochip mainly focuses on two directories:

A. Glass chip: Nucleotide fragments can be fixed and arranged in arrays on a general glass chip in a special way to prepare required gene biochip. Such a chip can not be popularized to applications in related fields; and the reasons are as follows:
i. The threshold of the glass chip technology is high and the cost is accordingly high so that general labs or examination units can not afford it.
ii. The fluorescent calorimetric reaction and the hybridization of the specimen (ribonucleic acid) which the glass chip uses require high quality and complex reaction steps; moreover, they require high technology, high cost of fluorescent marker (cy3 and cy5), and hard usage.
iii. The required scanner for analyzing the results of the fluorescent calorimetric reaction is a specific scanner whose cost is so high that a general lab can not afford it.

B. Nylon membrane chip: When preparing a biochip, the required nucleotide fragments are fixed on the nylon membrane. Comparing to the glass chip, a nylon membrane chip is easier to prepare; the threshold for the related experiment technology is lower; the experiment steps for the chemical calorimetric reaction is easier; and its reagent is cheaper. But, the required amount of specimen (ribonucleic acid) for the nylon membrane chip is larger and the analysis for the results of the chemical calorimetric reaction is not automatic so that the sensitivity and the accuracy are critically affected. Consequently, the nylon membrane chip is gradually replaced by the glass chip.

Besides, either the glass chip or the nylon membrane chip requires automatic spotting device. But the automatic spotting device costs quite high and is not easy to operate. Even those labs who can operate the device can not prepare the required chips independently which are prepared by some other biochemical companies. So, the technology for preparing the general gene biochip mentioned above does not fulfill the requirements of the user on actual applications.

### SUMMARY OF THE INVENTION

Therefore, the main purpose of the present invention is to effectively work out the functions and the applications of a gene biochip by a method for a gene biochip operation technology platform which is with low cost, easy operation and high efficiency.

Another purpose of the present invention is to conclusively popularize the practical applications of gene biochip on related fields.

To achieve the above purposes, the present invention is a method for a biochip detecting limited cells, comprising the following steps:

A. Choose a nylon membrane chip having nucleotide fragments arranged in a dot matrix way by using a manual spotter.

B. Naturally dry the chip by heat after spotting; and fix the nucleotide fragments by a rapid nucleic acid cross-linker so that a biochip is prepared.

C. Collect some normal whole blood as a specimen.

D. Extract the ribonucleic acid in the whole blood to be linearly amplified.

E. Synthesize required amount of cDNAs (Complementary Deoxyribonucleic Acid) by a reverse transcription; and label the cDNAs to obtain a marker as a probe.

F. Process labeling, hybridization and post-hybridization to the chip and the marker.

G. Process chemical color reaction to the chip and the marker after the post-hybridization.

H. And, automatically analyze the result image after the chemical color reaction.

Accordingly, by the above steps, a method for a gene biochip operation technology platform with low cost, easy operation and high efficiency is obtained so that the functions and the applications of the gene biochip can be effectively worked out and the practical applications of the gene biochip on related fields can be conclusively popularized.

### BRIEF DESCRIPTIONS OF THE DRAWINGS

The present invention will be better understood from the following detailed description of preferred embodiments of the invention, taken in conjunction with the accompanying drawings, in which:

FIG. 1 is a flowchart diagram according to the present invention;

FIG. 2 is a perspective view of the manual spotter according to the present invention;

FIG. 3 is an exploded view showing the structure of the manual spotter according to the present invention;

FIG. 4 is a view showing the results made from the statistical software for biochemistry according to the present invention;

FIG. 5 is a view showing the experimental results of K-ras mutant stably transfected adrenocortical cells according to the present invention;

FIG. 6 is a view showing the reaction of some cancer patients' blood to a specific genome on a technology platform of diagnostic chip for the nucleic acid in limited cells according to the present invention; and

Table. 1 is a table showing the diagnostic results come from the technology platform of diagnostic chip for the nucleic acid in limited cells which is confirmed by Real-Time PCR (Polymerase Chain Reaction) according to the present invention.

### DESCRIPTION OF THE PREFERRED EMBODIMENT

The following descriptions of the preferred embodiments are provided to understand the features and the structures of the present invention.

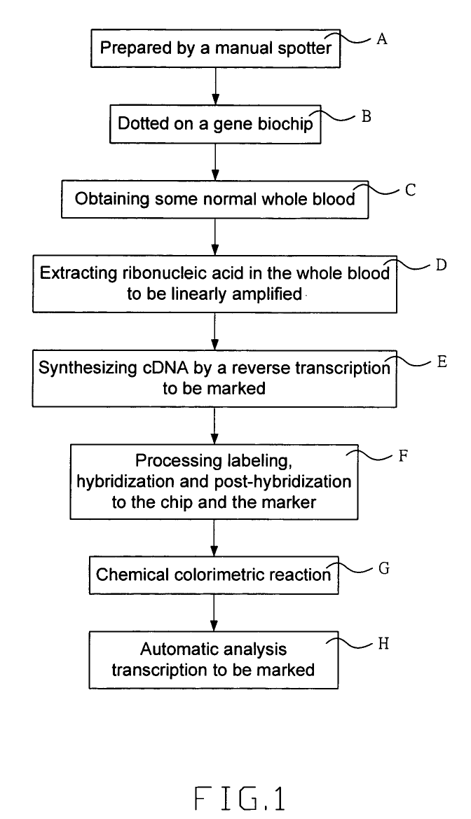

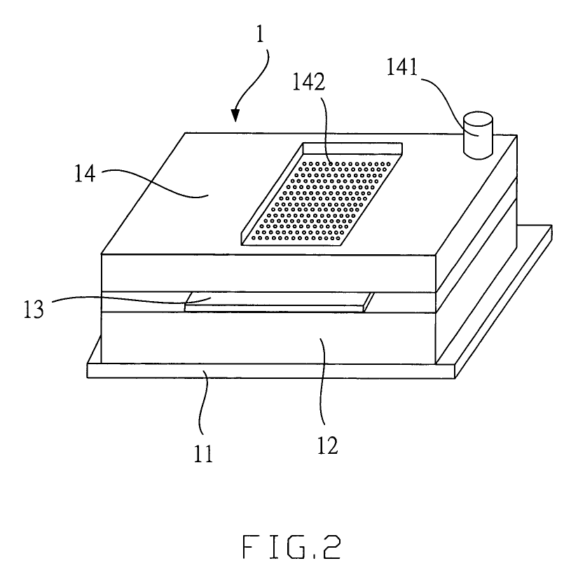

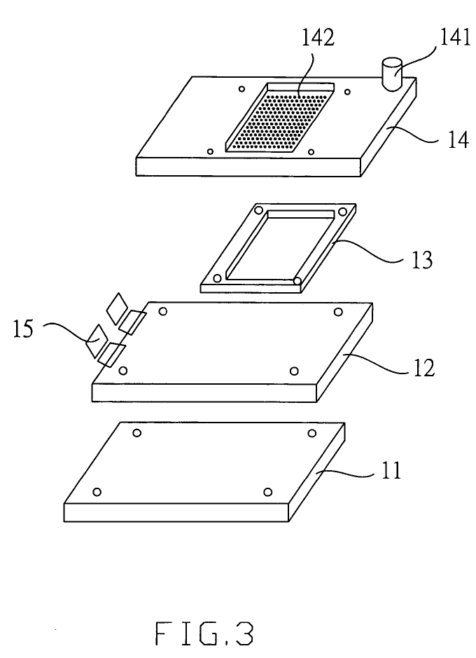

Please refer to FIG. 1 through FIG. 3, which are a flowchart diagram, a perspective view of the manual spotter and an exploded view showing the structure of the manual spotter, according to the present invention. As shown in the figures, the present invention is a method for a biochip detecting limited cells, comprising the following steps:

A. Obtain a nylon membrane chip having nucleotide fragments arranged in a dot matrix way by using a manual spotter, where each nucleotide fragment is obtained by soaking a nucleotide fragment with a di-distilled water to a size of 100 nl (nanoliter) with a length of 200 μM (micrometer) together with an interval of 1.5 mm (millimeter) between each two fragments. The manual spotter 1 comprises a base 11, a chip settlement layer 12 deposited on the base 11, a chip fixing layer 13 deposited on the chip settlement layer 12, and a dotting layer 14 deposited on the chip fixing layer 13. The chip settlement layer 12 and the dotting layer 14 are connected together at a side with a pivot 15 so that they can be opened at a proper position of the other side by an opening handle 141 of the dotting layer 14. The dotting layer 14 comprises at the center an area having a plurality of holes 142; the area is a square area of 4.5 cm (centimeter)×4.5 cm having 196 holes 142 (in 14 lines and 14 columns) each of whose inner diameter is 1.2 mm and is to be inserted with a trace suction tube (i.e. spotting nozzles) later.

The method for using the manual spotter comprises the following steps:
i. Open up the dotting layer 14 with the opening handle 141 of the manual spotter 1. Put the chip on the chip settlement layer 12.
ii. Close the dotting layer 14. The chip fixing layer 13 automatically fix the chip.
iii. Suck soak-made oligo-RNA (ribonucleic acid) with the trace suction tubes and directly insert the tubes into the holes 142, which are at the center of the dotting layer 14, to spot the oligo-RNA.

B. Naturally dry the chip by heat after spotting. And fix the nucleotide fragments on the nylon membrane chip by a rapid nucleic acid cross-linker so that a biochip is prepared. The nucleic acid cross-linker is processed with a crosslink in an energy of 1200 joule to fix the nucleotide fragments on the nylon membrane chip.

C. Collect some normal whole blood as a specimen. Only 1 c.c. is required to acquire 40 units of cDNAs (Complementary Deoxyribonucleic Acid) for detection; and only 4 units of cDNAs are required for each chip experiment.

D. Extract the RNA (ribonucleic acid) in the whole blood for linear amplification.

E. Synthesize required amount of cDNAs by a reverse transcription. And label the cDNAs to obtain a marker as a probe.

F. Process labeling, hybridization and post-hybridization to the chip and the marker. The time for labeling, hybriddization and post-hybridization is 24 hours.

G. Process chemical color reaction to the chip and the marker after the post-hybridization. During the post-hybridization, the time for detection is 30 minutes so that the calorimetric reaction in the post-hybridization is speeded up; and the color of the background will not be over-colorimetric and the false positive reaction can be reduced. The membrane washing which is before the blocking and the detection and is after the post-hybridization can be done with self-made washing buffer. And the membrane before the colorimetry can be processed by a self-made detection buffer.

H. Automatically analyze the result image by general density-analysis software after the chemical color reaction.

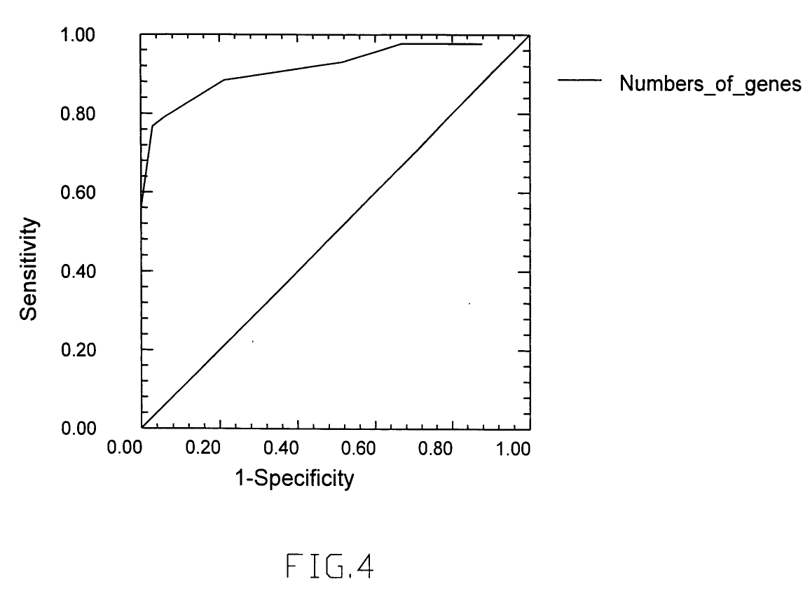

Please refer to FIG. 4, a statistical software for biochemistry (Statistical Package for the Social Sciences Ver. 10.0) is used to analysis the experiment results. From the Receiver Operating Characteristics curve obtained, concluded is that, by comparing the performance of each of the 22 genes on each biochip to that for a normal gene, if at least 11 genes, more than 50%, is in a status of twice performance to that of a normal gene, it is recognized as a positive (+) reaction; on the contrary, a negative (−) reaction. Accordingly, one of the most reasonable statistic can be obtained and the value for its sensitivity as well as its specificity is located at one of the most proper position, which becomes the standard for judging the data.

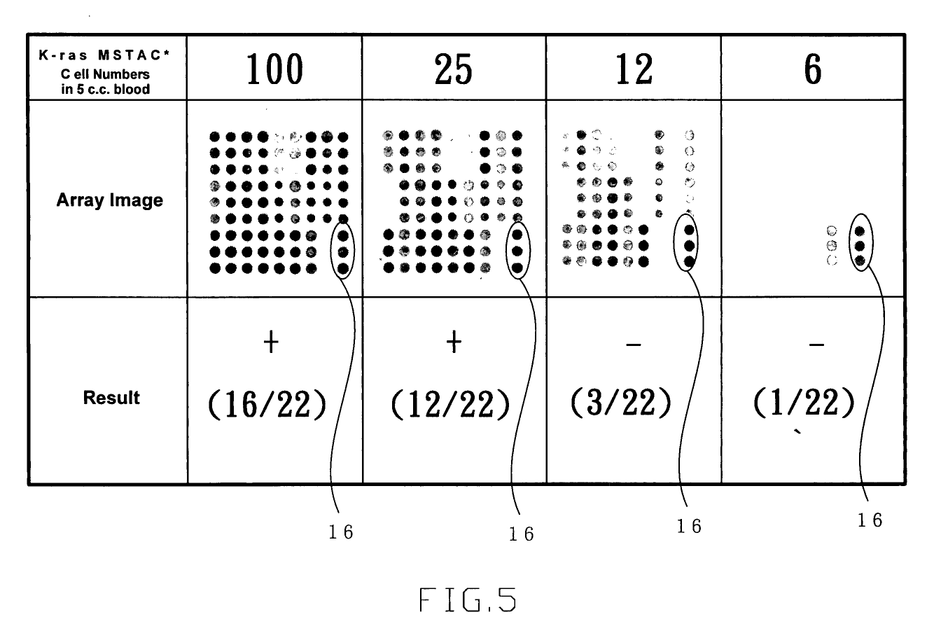

Please refer to FIG. 5. To ensure the possibility and the sensitivity of the method according to the present invention, an experiment in a glass container is processed. A pre-made K-ras mutant stably transfected adrenocortical cells (K-ras MSTAC) is added to a normal blood which is to be reacted with the detection biochip. Then, an analysis to the detection biochip is done with a positive (+) control which is a β-actin 16. The results show as what follows: There are twenty-two genes in every biochip. When there are twenty K-ras MSTAC cells in 1 c.c. of blood, there are sixteen genes in an excessive performance so that the reaction of the biochip is judged to be positive (+); in another word, there are 72.72%, more than 50%, of genes is in a positive reaction. When there are five K-ras MSTAC cells in 1 c.c. of blood, there are twelve genes in an excessive performance so that the reaction of the biochip is judged to be positive; in another word, there are 54.55%, more than 50%, of genes is in a positive reaction. But, when there are only two K-ras MSTAC cells in 1 c.c. of blood, there are only three genes in an excessive performance so that the reaction of the biochip is judged to be negative; in another word, there are only 13.64%, less than 50%, of genes is in a positive reaction. When the number of K-ras MSTAC cells in 1 c.c. of blood is down to one, only one gene left is in an excessive performance and, no doubt, the reaction of the biochip is judged to be negative. So, the method for a biochip detecting limited cells according to the present invention is workable that only more than five cells in 1 c.c. of blood are required to obtain a precise activetion analysis of the K-ras MSTAC cells in the blood with the detection biochip.

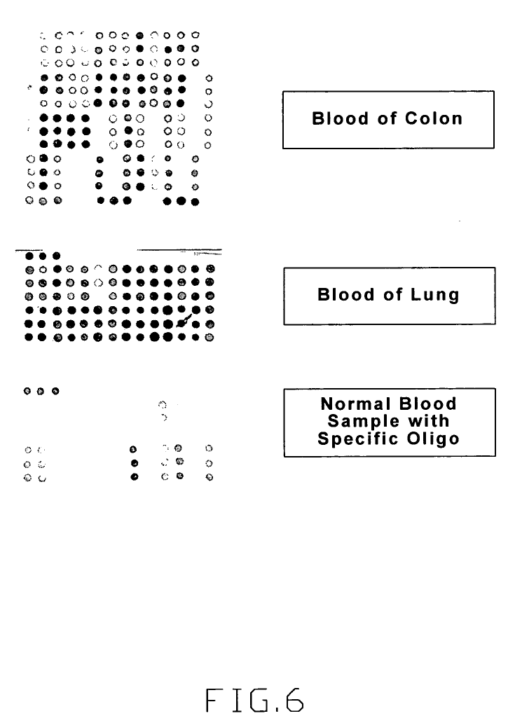

Please refer to FIG. 6 and Table. 1, which are a view showing the reaction of some cancer patients' blood to a specific genome on a technology platform of diagnostic chip for the nucleic acid in limited cells according to the present invention; and a table showing the diagnostic results come from the diagnostic chips with a technology platform for the nucleic acid in limited cells which is confirmed by Real-Time PCR according to the present invention. As shown in the figures, FIG. 6 shows the results of the RNA, which are extracted from some cancer patients' blood, reacting to the diagnostic chip according to the present invention. Table. 1 is a table showing the real-time PCR according to the present invention. Because the PCR is a technology for detecting the reaction of the RNA, the PCR can be applied to detect the reaction of a gene on the diagnostic chip according to the present invention to see whether it is identical to the amplification behavior of the same gene under PCR. By referring to Table. 1, the amplification reaction of the gene detected from the biochip according to the present invention shows the same amplification reaction as under PCR.

Consequently, the present invention comprises the following characteristics:

A. The Development of a manual spotter: In the past, the automatic fabrication machine was too expansive to be popularized and not easy for operation; and its pump was not stable. The nylon membrane is apt to be contaminated so that the sizes of the spots made by the traditional dot blotting are not of the same size. And the traditional dot blotting can not produce many spots of nucleotide fragments on the same nylon membrane at a time. To solve the above problems, the present invention develops a manual spotter with easy operation as well as the capability of blotting many spots of genes at the same time, so that the genes can be blotted in a matrix way to a size of 100 nl with an interval of 1.5 mm between each two dots.

B. Biochip preparation: To popularize proper applications of the biochip on related fields, the present invention chooses the nylon membrane instead of the glass membrane to solve the problems of high cost and high technology threshold. After obtaining a nylon membrane, required amount of nucleotide fragments are spotted on the chip in a matrix way by the manual spotter according to the present invention. The fragments are made by being soaked with a di-distilled water to a size of 100 nl with a length of 200 μM (micrometer). After the manual spotting, the chip is dried with heat. And, by a rapid nucleic acid cross-linker, the nucleotide fragments are processed with a crosslink by an energy of 1200 joule to be fixed on the nylon membrane chip and so a biochip is prepared.

C. Specimen treatment: Up to now, the required amount of the nucleic acid for a biochip experiment is great. Take the most common used glass biochip microarray for example, the value of A₂₆₀/A₂₈₀ of the nucleic acid (spectrophotometric absorbance ratio at 260 nm by a 280 nm wavelength, while ‘nm’ means ‘nanometer’) is strictly required to be above 1.8 and the amount of the nucleic acid has to reach mRNA 2 μg (total RNA 100 μg, while ‘mRNA’ means ‘messenger RNA’ and ‘μg’ means ‘microgram’). Because the specimen is not easy to obtain, the practical applications of the biochip is limited to the examination of microorganism and organ abundant in RNA only; and so the popularization and the development of the biochip are held back too.

So, the present invention improves the traditional experiment method to a novel method of treatment for the specimen. The RNA is linearly amplified to solve the problem of the limitation of specimen and to stabilize the amount of required cDNA synthesized by reverse transcriptional test. Even with tiny amount of RNA in the specimen, the reaction of the biochip is still available. Not only is the range for the quality requirement broadened (only 1.2 of A₂₆₀/A₂₈₀ is required) but also only 1 c.c. of whole blood is required to obtain 40 units of cDNA and only 4 units of cDNA is required for each biochip experiment.

D. The simplification of the experiment steps for labeling, hybridization and post-hybridization: All experiment steps are easy and the equipments are simple according to the present invention that only a temperature control machine is required to finish all the steps. The time for labeling, hybridization and post-hybridization is prolonged to 24 hours which further solves the problem of weak reaction for tiny amount of specimen. Besides, the time for detection during post-hybridization is prolonged from 5˜10 minutes to 30 minutes so that the calorimetric reaction is speeded up; the background color will not be over-colorimetric; and the False Positive reaction can be reduced.

In addition, the required reagents for the blocking buffer, the washing buffer and the detection buffer can be self-made with the following ingredients: [1] Blocking buffer—50 grams of blocking reagent; [2] Washing buffer—0.4% SDS (Sodium Dodecyl Sulphate) or 0.5×SSC (Sodium Chloride Sodium Citrate); and [3] Detection buffer—0.1M (mole/molar) Tris-HCl, 0.1M NaCl, or 50 mM (milli-mole/molar) MgCl₂●6H₂O. And, the detection and the secondary antibody can be recycled. Therefore, common laboratories and related authorities can finish all the experiment steps independently.

E. Automatic analysis: The image acquired from the experiment can be automatically analyzed by density analysis software, which solves the problem of the inaccurate judgment to the experimental results of the calorimetric reaction in the past. The density analysis software can be Alpha Ease FC Stand Alone V.3.1.2 or GeneTAC™ Integrator Version 3.3. And the software is easy to operate and is low in cost. The results obtained can also be further utilized by related software of biological information according to the user's requirements.

To sum up, the method for a biochip detecting limited cells according to the present invention, not only surmounts the difficulty of popularizing the traditional chip experiment and make the gene biochip be able to be practically applied in related fields; but also practical applications of related researches or examinations can be run more smoothly, such as the analysis of the function and the behavior of a gene, the analysis of a gene mutation, the differential gene screening, the transcription factors searching, the gene study and invention of a new medicine, the clinical disease examination, the treatment follow-up, the food diagnostics, the agricultural product improvement and research, etc.

The preferred embodiments herein disclosed are not intended to unnecessarily limit the scope of the invention. Therefore, simple modifications or variations belonging to the equivalent of the scope of the claims and the instructions disclosed herein for a patent are all within the scope of the present invention.

What is claimed is:

1. A method for a biochip detecting limited cells, comprising steps of:
a. obtaining a nylon membrane chip having nucleotide fragments arranged in a dot matrix way by using a manual spotter;
b. naturally drying by heat after spotting, and firmly depositing said nucleotide fragments by a rapid nucleic acid cross-linker to prepare a biochip;
c. collecting some normal whole blood;
d. extracting ribonucleic acid in said whole blood to be linearly amplified;
e. synthesizing required amount of cDNAs (Complementary Deoxyribonucleic Acid) by a reverse transcription, and labeling said cDNAs to obtain a marker;
f. processing labeling, hybridization and post-hybridization to said chip and said marker;
g. processing chemical color reaction to said chip and said marker after said post-hybridization; and
h. automatically analyzing the result image after said chemical color reaction.

2. The method according to claim 1, wherein said manual spotter comprises a base; a chip settlement layer deposited on said base; a chip fixing layer deposited on said chip settlement layer; and a dotting layer deposited on said chip fixing layer.

3. The method according to claim 1, wherein said chip settlement layer and said dotting layer are connected together at a side with a pivot.

4. The method according to claim 1, wherein said dotting layer comprises an opening handle.

5. The method according to claim 1,
wherein said dotting layer comprises at the center an area having a plurality of holes;
wherein said area is a square area of 4.5 cm (centimeter) multiplied by 4.5 cm having 196 holes; and
wherein each said hole comprises an inner diameter of 1.2 mm (millimeter) and the distance between every two adjacent said holes is 3 mm.

6. The method according to claim 1, wherein said nucleotide fragments are obtained by soaking by di-distilled water to a size of 100 nl (nanoliter) with a length of 200 μM (micrometer) in a dot matrix way on said chip.

7. The method according to claim 1, wherein said nucleotide fragments of a size of 100 nl are arranged by said manual spotter in a dot matrix way with an interval of 1.5 mm (millimeter) between each two adjacent said nucleotide fragments.

8. The method according to claim 1, wherein said rapid nucleic acid cross-linker firmly deposit said nucleotide fragments on said nylon membrane in a cross-link way with an energy of 1200 joule.

9. The method according to claim 1, wherein only 1 c.c of said normal whole blood is required for detection.

10. The method according to claim 1, wherein the time for said labeling and said hybridization is 24 hours.

11. The method according to claim 1, wherein, on processing said post-hybridization, the time for detection is 30 minutes.

12. The method according to claim 1, wherein said result image in step h is analyzed by density analysis software selected from a group consisting of Alpha Ease FC Stand Alone and GeneTAC™ Integrator.
